SEQUENCE LISTING (1 sequence)

SEQ ID NO: 1 - RNA, 1527 nt, Streptomyces sp.
  source 1..1527: mol_type = unassigned RNA
acggagagtttgatcctggctcaggacgaacgctggcggcgtgcttaacacatgcaagtcgaacgatgaacttccttcgggaggggattagtggcgaacgggtgagtaacacgtgggcaatctgccctgcactctgggacaagccctggaaacggggtctaataccggatacgacacgggatcgcatgatcttccgtgtggaaagctccggcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggaatcgtcgaaggtgggactggcgattgggacgaagtcgtaacaaggtagccgtaccggaaggtgcggctggatcacctccttt

(19) United States
(12) Patent Application Publication
(10) Pub. No.: US 2026/0344665 A1
(43) Pub. Date: Nov. 5, 2026

(54) FUNGICIDAL COMPOUND

(71) Applicant: SYNGENTA CROP PROTECTION AG, Basel (CH)

(72) Inventors: Stephane BIERI, Stein (CH); Philip SIDEBOTTOM, Bracknell, Berkshire (GB); Dimitrios PAPASOTIRIOU, Bracknell, Berkshire (GB); Peter VAN DE VONDERVOORT, Echt (NL); Leon COULIER, Echt (NL); Dianne IRWIN, Bracknell, Berkshire (GB)

(73) Assignee: SYNGENTA CROP PROTECTION AG, Basel (CH)

(21) Appl. No.: 19/118,467

(22) PCT Filed: Oct. 5, 2023
(86) PCT No.: PCT/EP2023/077603

§ 371 Date: Apr. 4, 2025

(30) Foreign Application Priority Data
Oct. 7, 2022 (EP) 22200224.8

(51) Int. Cl. A01N 43/08; A01N 63/28; A01P 3/00; A01P 21/00; C12N 1/20; C12P 19/60; C12R 1/465

(52) U.S. Cl. CPC A01N 43/08; A01N 63/28; A01P 3/00; A01P 21/00; C12N 1/20; C12P 19/60; C12R 2001/465

(57) ABSTRACT

The present invention relates to a compound of Formula (I) and a composition containing it, a process of producing the compound and a method of using the compound and the compositions to prevent or control fungi in plants and in agriculture.

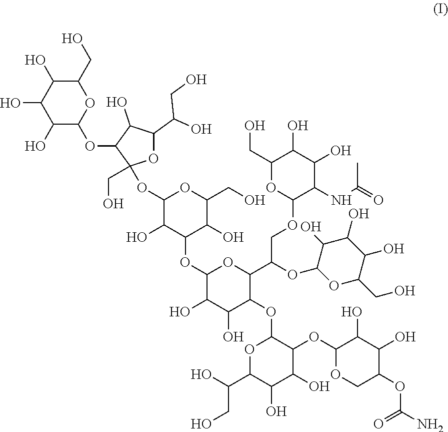 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]

The present invention relates to a novel compound which has fungicidal activity. The invention also relates to compositions which comprise the compound, to a process of preparation of the compound and to the use of the compound or the compositions in agriculture or horticulture for preventing or controlling fungal infestation of plants, harvested food crops, seeds or non-living materials.

### BACKGROUND

Fungicides are widely used in agriculture to protect plants against damage caused by fungi. Fungicides may be from chemical origin or biological origin. Biological fungicides may for instance be from microbial origin or can be a plant extract. Known microorganisms that produce antibiotics against fungi are for instance actinomycetes, for instance Streptomyces sp. A very well-known species is Streptomyces natalensis that produces the antifungal compound natamycin, which is used in food and crop protection. In U.S. Pat. No. 5,356,624 a Streptomyces rimosus strain is disclosed that was found active against several wood-degrading fungi. In WO2022/038180 new Streptomyces sp. are disclosed that produce several known antifungal compounds such as streptimidone, natamycin (pimaricin), or albofungin. The extracts of these bacterial strains were found active against well-known plant pests such as Fusarium graminearum, Zymoseptoria tritici, and Puccinia striiformis.

Due to development of resistance by fungi against fungicides, regulations by governments and societal pressure there is a continuous need to look for new compounds that have fungicidal activity from biological origin.

### SUMMARY

The present invention relates to a compound comprising a molecular formula according to C₅₃H₉₀N₂O₄₄, preferably having a molecular mass of 1458.487 g further characterised by the NMR spectra listed in Table 1 and Table 2, or a salt thereof.

The invention further relates to a compound of structural Formula (I):

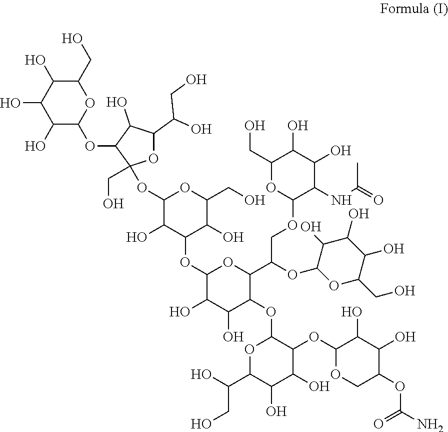 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]

or a salt thereof.

In a second aspect the invention relates to a composition comprising a compound according to the present invention and a microorganism that is able to produce a compound according to the present invention.

In a third aspect the invention relates to a process for producing the compound or a composition according to the present invention, comprising cultivating a microorganism in a suitable fermentation medium under conditions that allow production of the compound.

In a fourth aspect the present invention relates to a method for controlling or preventing infestation of a plant by a phytopathogenic microorganism, wherein an effective amount of the compound or a composition according to the invention is applied to the plant, to a part thereof or a locus thereof.

According to a fifth aspect of the invention, there is provided the use of a compound or compositions according to the present invention as a pesticide, preferably a fungicide, and/or as a priming agent. According to this aspect of the invention, the use excludes methods for the treatment of the human or animal body by surgery or therapy.

### DETAILED DESCRIPTION

According to the present invention, there is provided a compound comprising a molecular formula according to C₅₃H₉₀N₂O₄₄ further characterised by the NMR spectra listed in Table 1 and Table 2, or a salt thereof. The compound comprising a molecular formula of C₅₃H₉₀N₂O₄₄ has a molecular mass of 1458.487 g. Preferably the compound according to the present invention has a solubility at a pH 7 of more than >10′000 ppm, and/or a solubility in DMSO of above >9772 ppm.

Preferably, the compound according to the present invention is an isolated compound. The wording ‘isolated’ with reference to the compound means that the compound has been isolated from it's native environment. Preferably, the compound according the present invention is an oligosaccharide.

The compound according to the present invention may be a compound having a structural formula of Formula (I):

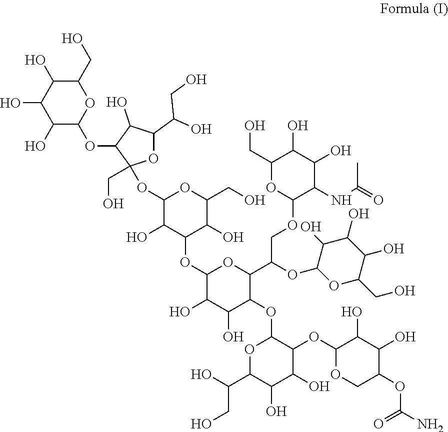 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]

or a salt thereof.

In one aspect, the present invention relates to a composition comprising the compound according to the present invention and a microorganism that is able to produce a compound as disclosed herein. The composition comprising the compound according to the present invention and a microorganism that is able to produce a compound according to the present invention is applied to plants or to parts thereof to cure or protect plants against diseases caused by phytopathogenic microorganisms such as fungi, bacteria or viruses.

Surprisingly, it has now been found that the compound and/or composition according to invention has an advantageous level of biological activity for curing or protecting plants against diseases that are caused by infestations of phytopathogenic microorganisms such as fungi, bacteria or viruses. Surprisingly, the compound and/or composition according to the present invention has an advantageous fungicidal activity against various phytopathogenic fungi such as Zymoseptoria tritici, Puccinia recondita f. sp. tritici, Magnaporthe grisea, Blumeria graminis f.sp. tritici and/or Parastagonospora nodorum.

The composition according to the present invention is a non-naturally occurring composition.

Preferably, the compound and/or composition according to the present invention has/have fungicidal activity. Accordingly, the compound and/or composition according to the present invention preferably are/is a fungicide.

The term “compound having fungicidal activity” or “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

Preferably, the microorganism able produce a compound according to the present invention, for instance in the composition as disclosed herein is a bacterium of the genus Streptomyces sp., preferably the bacterium is a Streptomyces chrestomyceticus, S. rimosus, or S. albofaciens, S. paromomycinus, or S. monomicini. Preferably, the composition comprises Streptomyces sp. Saigon413 deposited with the Westerdijk Institute under accession number CBS149411. Preferably, the microorganism is a Streptomyces sp., preferably Streptomyces sp. Saigon413 deposited with the Westerdijk Institute under accession number CBS149411, wherein the Streptomyces sp. has a 16S RNA sequence which has at least 98%, preferably at least 98.2%, 98.4%, 98.6%, 98.8%, preferably at least 99%, 99.2%, 99.4%, 99.5%, 99.6%, 99.7%, 99.8% preferably at least 99.9%, or has 100% identity to SEQ ID NO: 1.

Preferably, the microorganism able to produce a compound according to the present invention comprises a genome sequence which has at least 89%, 90%, 91%, 92%, 93%, 94%, 95%, 96%, 97%, 98%, 99% identity or 100% identity to the whole genome of Streptomyces sp. Saigon413 deposited with the Westerdijk Institute under accession number CBS149411

As used herein, the terms “percent identity,” and “percent identical” refer to the relatedness of two or more nucleotide or amino acid sequences, which may be calculated by (i) comparing two optimally aligned sequences over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100 percent to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present invention, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100 percent.

Also disclosed herein is a microorganism which is a Streptomyces sp. Saigon413 deposited with the Westerdijk Institute under accession number CBS149411.

It was surprisingly found that Streptomyces sp. Saigon413 deposited with the Westerdijk Institute under accession number CBS149411 has advantageous properties as compared to Streptomyces species known in the art.

The compound of the present invention, or a composition comprising the compound of the present invention can be used in the agricultural sector and related fields of use, e.g., as active ingredients for controlling plant pests. The compound according to the present invention is distinguished by excellent activity at low rates of application such as from 5 to 300 ppm, for instance 10 to 250 ppm, for instance 20 to 200 ppm, by being well tolerated by plants and by being environmentally safe. It has very useful curative, preventive and systemic properties and can be used for protecting numerous plants. The compound of the present invention can be used to inhibit or destroy pests that occur on plants or parts of plants (fruit, blossoms, leaves, stems, tubers, roots) of different crops of plants, while at the same time protecting also those parts of the plants that grow later.

The compound according to the present invention or a composition comprising the compound according to the present invention can be used as such or formulated with an auxiliary, preferably an agricultural-acceptable auxiliary. Known formulations in the art are for instance emulsifiable concentrates, coatable pastes, sprayable or dilutable solutions or suspensions, powders, dusts, granulates and encapsulations.

Accordingly, in one embodiment a composition comprising a compound according to the present invention as disclosed herein further comprises an auxiliary. Preferably the auxiliary is an agricultural-acceptable auxiliary.

Suitable auxiliaries are known in the art, and include for example solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers include, for example water, organic solvents, oils of vegetable or animal origin, cyclic and aromatic hydrocarbons, alcohols, esters, fatty acids, a glycol or any other suitable liquid carrier known in the art. The solvent or liquid carrier may be water or DMSO.

Suitable solid carriers include, for example, talc, titanium dioxide, pyrophyllite clay, silica, attapulgite clay, kieselguhr, chalk, diatomaceous earth, lime, calcium carbonate, bentonite clay, fuller's earth, cotton seed hulls, wheat flour, soybean flour, pumice, wood flour, walnut shell flour and lignin.

An adjuvant may be a surface-active agent, crystallisation inhibitor, viscosity modifier, suspending agents, spray droplet modifiers, pigments, antioxidants, foaming agents, anti-foaming agents, light-blocking agents, compatibilizing agents, sequestering agents, neutralising agents and buffers, corrosion inhibitors, dyes, odorants, spreading agents, penetration aids, micronutrients, emollients, lubricants and sticking agents.

A composition as disclosed herein preferably is an agricultural-acceptable composition.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the condensation product of formaldehyde with naphthalene sulphonate, an alkylarylsulphonate, a lignin sulphonate, a fatty alkyl sulphate, and ethoxylated alkylphenol and an ethoxylated fatty alcohol.

A composition comprising a compound according to the present invention typically comprises 0.5 to 95 w/w % of active ingredient such as from 1 to 90 w/w %, such as from 2 to 80 w/w %, such as from 5 to 60 w/w %. The compound according to the present invention may be the sole active ingredient in a composition as disclosed herein. In one embodiment, a composition comprising a compound of the present invention further comprises at least one additional active ingredient. An active ingredient as defined herein is an ingredient that has fungicidal and/or insecticidal and/or herbicidal activity or has activity as plant growth regulator. The compound of the present invention or a composition as disclosed herein may be admixed with one or more additional ingredients having pesticidal activity known in the art, such as fungicides, insecticides, herbicides, bactericides, acaricides, nematicides and/or the additional ingredient comprises plant growth regulators where appropriate. The one or more additional ingredients having pesticidal activity may be from biological or chemical origin. Pesticidal agents are referred to herein using their common name are known, for example, from “The Pesticide Manual”, 19th Ed., British Crop Protection Council 2021.

An additional ingredient having pesticidal activity, for instance fungicidal activity may result in an unexpected synergistic activity. Accordingly, a composition comprising a compound according to the present invention and an additional active ingredient, for instance malonomicin, may show a synergistic effect. A synergistic effect occurs whenever the action of an active ingredient combination is greater than the sum of the actions of the individual components. The action to be expected E for a given active ingredient combination obeys the so-called COLBY formula and can be calculated as follows (COLBY, S. R. “Calculating synergistic and antagonistic responses of herbicide combination”. Weeds, Vol. 15, pages 20-22; 1967):
ppm=milligrams of active ingredient (=a.i.) per liter of spray mixture
X=% action by active ingredient A) using p ppm of active ingredient
Y=% action by active ingredient B) using q ppm of active ingredient.

According to COLBY, the expected (additive) action of active ingredients A)+B) using p+q ppm of active ingredient is:

$$E = X ⋆ Y - \frac{X \cdot Y}{100} .$$

If the action actually observed (O) is greater than the expected action (E), then the action of the combination is super-additive, i.e. there is a synergistic effect. In mathematical terms, synergism corresponds to a positive value for the difference of (O-E). In the case of purely complementary addition of activities (expected activity), said difference (O-E) is zero. A negative value of said difference (O-E) signals a loss of activity compared to the expected activity.

The additional ingredients having pesticidal activity and/or which is a plant growth regulator may be combined with a composition of the invention and used in a method of the invention and applied simultaneously or sequentially with a composition of the invention. When applied simultaneously, these further ingredients may be formulated together with the compositions of the invention or mixed in, for example, a spray tank. As an alternative to directly admixing these further ingredients having pesticidal activity, the components may be used in separate fungicidal, insecticidal or herbicidal applications as part of a programme of fungal, insect or herbal control spread over part or all of a growing season.

The at least one additional ingredient having pesticidal activity and/or which is a plant growth regulator may be any suitable known fungicide, insecticide, herbicide and/or plant growth regulator. The at least one additional ingredient having pesticidal activity and/or plant growth regulator may be from chemical origin or biological origin, for instance from plant or microbial origin. The at least one additional ingredient having pesticidal activity in a composition as disclosed herein may be produced by a microorganism able to produce a compound according to the present invention.

In addition, the composition of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The compound and/or composition according to the present invention may induce resistance of a plant by a priming mechanism. Priming is a mechanism which leads to a physiological state that enables plants to respond more rapidly and/or more robustly after exposure to biotic or abiotic stress as described for instance in review article: P. Aranega-Bou et. al. Priming of plant resistance by natural compounds. Hexanoic acid as a model. Front. Plant. Sci. 1, October 2014.

In one embodiment the composition according to the present invention further comprises cyclothiazomycin C (CtmC), streptimidone and/or malonomicin.

The structure of cyclothazomycin C is disclosed on p. 3 of WO2015191789 and can be produced as disclosed in Example 4 of WO2015/191789.

Malonomicin (sometimes spelt ‘malonomycin’) is {[(2S)-2-amino-3-hydroxypropanoyl]amino} {2-[(5S)-5-(aminomethyl)-4-hydroxy-2-oxo-2,5-dihydro-1H-pyrrol-3-yl]-2-oxoethyl} malonic acid and has a structural formula of Formula II

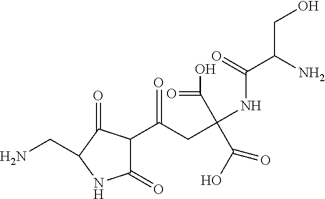 [SMILES: NCC1NC(=O)C(C(=O)CC(NC(=O)C(N)CO)(C(=O)O)C(=O)O)C1=O]

Malonomicin can be produced as disclosed in Example 1 of WO2006/078939. Malonomicin may also be prepared according to the method disclosed in Example I A and B in EP 1860939 or according to Law et al, 2018 (Nature Catalys is | VOL 1 | DECEMBER 2018 | 977-984).

Streptimidone is a known compound of Formula III:

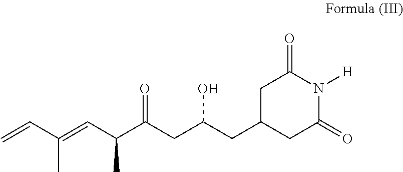 [SMILES: C=C/C(C)=C/[C@H](C)C(=O)C[C@H](O)CC1CC(=O)NC(=O)C1]

Streptimidone can be synthesised following the method disclosed in Kondo, H., Oritani, T., and Kiyota, H. Synthesis and antifungal activity of the four stereoisomers of streptimidone, a glutarimide antibiotic from Streptomyces rimosus forma paromomycinus Eur. J. Org. Chem. (20), 3459-3462 (2000). In one embodiment, the active ingredients cyclothiazomycin C, streptimidone and/or malonomicin are produced by the microorganism able to produce compound according to the present invention as defined herein above.

The present invention also relates to a composition comprising a compound having molecular formula C₅₃H₉₀N₂O₄₄, according to the present invention, and malonomicin. It was surprisingly found that a composition comprising compound having molecular formula according to C₅₃H₉₀N₂O₄₄ according to the present invention, preferably a compound according to Formula (I) and malonomicin can exhibit an unexpected synergistic fungicidal effect. A surprising synergistic fungicidal effect of a composition comprising a compound according to the present invention, and malonomicin was for instance found against Puccinia recondita.

A composition comprising the compound of the present invention and at least one additional active ingredient, for instance malonomicin, is preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight. A composition comprising a mixture of a compound according to the present invention and malonomicin is in a mixing ratio of from 1000:1 to 1:1000, preferably from 500:1 to 1:500, preferably from 450:1 to 1:300, preferably from 400:1 to 1:150, preferably from 350:1 to 1:100, or from 100:1 to 1:80. The mixtures as described above can be used in a method for controlling pests, such as phytopathogenic microorganisms, which comprises applying a composition comprising a mixture as described above to the pests or their environment with the exception of a method for treatment of the human or animal body by surgery or therapy and diagnostic methods practised on the human or animal body.

A composition comprising the compound of the invention and one or more additional ingredients having pesticidal activity or a plant growth regulator as described above can be applied, for example, in a single “ready-mix” form, in a combined spray mixture composed from separate formulations of the single active ingredient components, such as a “tank-mix”, and in a combined use of the single active ingredients when applied in a sequential manner, i.e., one after the other with a reasonably short period, such as a few hours, days or weeks.

In one aspect, the present invention relates to a process for producing the compound or composition according to the present invention comprising cultivating a microorganism in a suitable fermentation medium under conditions that allow production of the compound or composition. A microorganism that is fermented in a process as disclosed herein is a microorganism able to produce the compound according to the present invention, is disclosed herein above.

Cultivating a microorganism for producing the compound or composition according to the present invention in a suitable fermentation medium is known to a person skilled in the art. The microorganism may be fermented under aerobic or anaerobic conditions. A microorganism belonging to Streptomyces sp. is typically cultivated under aerobic conditions. A suitable fermentation medium comprises nutrients, such as a suitable carbon source such as cane or sugar beet molasses, polysaccarides, flour, starch, sugar or glucose, and a suitable nitrogen source, for instance casein hydrolysate, tryptone, ammonium sulphate, ammonia, yeast extract, peptone or urea, peptides or amino acids. The process for producing a compound according to the present invention may be performed in a batch, fed-batch or continuous culture.

In one embodiment the process comprises producing a compound or a composition comprising the compound according to the present invention and a microorganism able to produce the compound according to the present invention. A composition comprising the compound according to the present invention and a microorganism able to produce the compound according to the present invention may be a fermentation broth. A microorganism able to produce the compound according to the present invention in the process of the invention may be able to produce further active ingredients as defined herein above, for instance cyclothiazomycin C, streptimidone and/or malonomicin.

The process according to the present invention may further comprise a step of recovering the compound or composition according to the present invention. The compound according to the present invention may be recovered by suitable methods known in the art, for instance via crystallization or chromatography, eg HPLC. Recovering the compound according to the present invention may further comprise a step of purifying the compound.

The process for producing a compound according to the present invention may further comprise a step of formulating the compound into a suitable formulation or composition as defined herein above.

In one further aspect, the present invention relates to a method for controlling or preventing infestation of a plant, plant propagation material, locus and/or harvested food crops by a phytopathogenic microorganism, by treating a plant, plant propagation material, locus and/or harvested food crops and/or, wherein an effective amount of the compound according to the present invention or a composition according to the present invention, is applied to the plant, to a part thereof, the plant propagation material, the locus and/or harvested food crops.

Applying an effective amount of the compound of the present invention in a method for controlling or preventing investation of a plant comprises applying from 5 g to 5 kg of the compound of the invention (active ingredient (a.i.) per hectare (ha), preferably from 10 g to 1 kg a.i./ha, most preferably from 20 g to 600 g a.i./ha.

When the compound or composition of the present invention is used for treating seeds, rates of 0.001 to 50 g of the compound of the invention per kg of seed, preferably from 0.01 to 10 g per kg of seed are generally sufficient.

Suitably, a compound of the invention or a composition comprising the compound according to the present invention is applied either preventative, meaning prior to disease development or curative, meaning after disease development.

Phytopathogenic microorganisms that are affected by the compound of the invention are fungi and fungal vectors of disease as well as phytopathogenic bacteria and viruses. Phytopathogenic microorganisms in a method according to the present invention include the following fungi and fungal vectors of disease and phytopathogenic bacteria:

Absidia corymbifera, Albugo candida, Alternaria spp. including A. solani, Aphanomyces spp, Ascochyta spp, Aspergillus spp. including A. flavus, A. fumigatus, A. nidulans, A. niger, A. terrus, Aureobasidium spp. including A. pullulans, Bacillus subtilis, Blastomyces dermatitidis, Blumeria graminis, Blumeria graminis f.sp. tritici, Blumeriella jaapii, Botryosphaeria spp. including B. dothidea, B. obtusa, Botrytis spp. including B. cinerea, Bremia lactucae, Cadophora gregata, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, C. beticola, C. kikuchii, C. sojina, Cercosporidium personatum, Cladosporium spp, Clarireedia homoeocarpa, Clavibacter spp, Claviceps purpurea, Coccidioides immitis, Cochliobolus spp, Colletotrichum spp. including C. dematium, C. lindemuthianum, C. musae, C. orbiculare, C.truncatum, Corynespora cassiicola, Cryptococcus neoformans, Diaporthe spp, Dickeya zeae, Didymella spp, Drechslera spp, Elsinoe spp, Epidermophyton spp, Eremothecium gossypiim, Erwinia spp. including E. amylovora, E. carotovora, Erysiphe spp. including E. cichoracearum, E. necator, Eutypa lata, Fusarium spp. including F. culmorum, F. graminearum, F. langsethiae, F. moniliforme, F. oxysporum, F.poae, F. proliferatum, F. pseudograminearum, F. sacchari, F. sambucinum, F. subglutinans, F. solani, F. sporotrichioides, F. tricinctum, F. virguliforme, Gaeumannomyces graminis, Gibberella spp. including G. avenacea, G. fujikuroi, G. intricans, G. moniliformis, G. zeae, Gloeodes pomigena, Gloeosporium musarum, Glomerella cingulate, Golovinomyces cichoracearum, Gymnosporangium juniperi-virginianae, Guignardia bidwellii, Gymnosporangium juniperi-virginianae, Helminthosporium spp, Hemileia spp, Histoplasma spp. including H. capsulatum, Hyaloperonospora parasitica, Kabatiella zeae, Laetisaria fuciformis, Leptographium lundbergii, Leveillula taurica, Lophodermium seditiosum, Microdochium majus, Microdochium nivale, Microsporum spp, Monilinia spp. including M. fructicola, Monographella spp including M. nivalis, Mucor spp, Mycosphaerella spp. including M. arachidis, M. fijiensis, M. graminicola, M. pomi, Nakataea oryzae, Neopseudocercosporella spp, Oculimacula spp, Oncobasidium theobromaeon, Ophiostoma spp, Pantoea stewartia, Paracoccidioides spp, Parastagonospora nodorum, Pectobacterium spp, Penicillium spp. including P. digitatum, P. italicum, Petriellidium spp, Peronosclerospora spp. including P. maydis, P. philippinensis and P. sorghi, Peronospora spp including P. destructor, Phaeosphaeria nodorum, Phakopsora pachyrhizi, Phellinus igniarus, Phialophora spp, Phlyctema vagabunda, Phoma spp, Phomopsis viticola, Phyllachora pomigena, Phyllosticta spp, Physoderma maydis, Phytophthora spp. including P. capsica, P. infestans, Plasmodiophora brassicae, Plasmopara spp. including P. halstedii, P. viticola, Plenodomus spp, Pleospora spp., Podosphaera spp. including P. leucotricha, Polymyxa graminis, Polymyxa betae, Pseudocercospora fijiensis, Pseudocercosporella herpotrichoides, Pseudomonas spp. including P. syringae, Pseudoperonospora spp. including P. cubensis, P. humuli, Pseudopeziza tracheiphila, Pseudopyrenochaeta lycopersici, Puccinia spp. including P. hordei, P. recondita, P. striiformis, P. triticina, Pyrenopeziza spp, Pyrenophora spp, Pyricularia spp. including P. oryzae, Pythium spp. including P. ultimum, Ralstonia solanacearum, Ramularia spp, Rathayibacter spp, Remotididymella destructiva, Rhizoctonia spp, Rhizomucor pusillus, Rhizopus arrhizus, Rhynchosporium spp, Robbsia andropogonis, Sarocladium oryzae, Scedosporium spp. including S. apiospermum and S. prolificans, Schizothyrium pomi, Sclerophthora macrospora, Sclerotinia spp. including S. sclerotiorum, Sclerotium spp, Septoria spp, including S. nodorum, S. tritici, Setosphaeria turcica, Sphaerotheca macularis, Sphaerotheca fusca (Sphaerotheca fuliginea), Spiroplasma kunkelii, Sporothorix spp, Stagonospora nodorum, Stagonosporopsis cucurbitacearum, Stemphylium spp, Stenocarpella macrospora, Stereum hirsutum, Streptomyces spp, Thanatephorus cucumeris, Thielaviopsis basicola, Tilletia spp, Tranzschelia discolor, Trichoderma spp. including T. harzianum, T. pseudokoningii, T. viride, Trichophyton spp, Typhula spp, Uncinula necator, Urocystis spp, Uromyces spp, Ustilago spp, Venturia spp. including V. inaequalis, Verticillium spp, Wilsonomyces carpophilus, or Xanthomonas spp, including X. oryzae and X. campestris, Xylella spp, Zymoseptoria tritici.

Phytopathogenic microorganisms that are found to be surprisingly affected by the compound and/or composition according to the present invention, are fungi, for instance fungi belonging to Zymoseptoria tritici, Puccinia recondita f. sp. tritici, Magnaporthe grisea, or Blumeria graminis f.sp. tritici.

Any suitable plant, plant propagation material, locus or food crop may be treated in a method according according to the present invention as defined herein.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits. Germinated plants and young plants which are to be transplanted after germination or after emergence from the soil, may also be mentioned. These young plants can be protected before transplantation by a total or partial treatment by immersion. The term “plant propagation material” is understood to denote generative parts of the plant, such as seeds, which can be used for the multiplication of the latter, and vegetative material, such as cuttings or tubers, (for example potatoes), roots, fruits, bulbs, rhizomes or parts of plants.

The term plants involve “useful plants” or “crops”. The wording “Useful plants” and “crops” are used interchangeably herein. “Useful plants” and “crops” comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes. The term “plants” also includes wood crops, such as pine trees, or woody plants.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Preferably the plant, plant propagation material or food crop is rice, wheat, corn, soya, or banana. The wording soya as herein includes soybean.

Controlling or preventing in a method of the invention means reducing infestation by phytopathogenic microorganisms, especially fungi, to such a level that an improvement is demonstrated.

A preferred method of controlling or preventing an infestation of crop plants by phytopathogenic microorganisms, especially fungi, is foliar application of the compound and/or composition according to the present invention. The frequency of application and the rate of application will depend on the risk of infestation by the corresponding pathogen or insect. Alternatively, the compound and/or composition according to the present invention can penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compound according to the present invention may also be applied to seeds (coating) by impregnating the seeds or tubers either with a liquid formulation of the fungicide or coating them with a solid formulation.

It is also possible to use the compound of the invention as a dressing agent for the treatment of plant propagation material, e.g., seed, such as fruits, tubers or grains, or plant cuttings, for the protection against fungal infections as well as against phytopathogenic fungi occurring in the soil. The propagation material can be treated with the compound and/or or composition according to the invention before planting: seed, for example, can be dressed before being sown. Disclosed herein are such methods of treating plant propagation material and the plant propagation material so treated.

In another aspect the invention relates to the use of a compound according to the present invention or a composition comprising the compound according to the present invention as a pesticide, preferably as a fungicide and/or as a priming agent. The features related to the compound according to the present invention and the composition comprising a compound according to the present invention are as disclosed herein above. Accordingly, the present invention relates to a method for using a compound according to the present invention as a pesticide, preferably as a fungicide and/or as a priming agent.

### FIGURES

FIG. 1. 1D ¹H NMR spectrum of the compound of the invention in D₂O at 600 MHz

FIG. 2. 1D ¹³C NMR spectrum of the compound of the invention in D₂O at 600 MHz

FIG. 3. 2D Dept Edited ¹H-¹³C HSQC NMR Spectrum of the compound of the invention in D₂O at 600 MHz showing the positive (CH) signals

FIG. 4. 2D Dept Edited ¹H-¹³C HSQC NMR Spectrum of the compound of the invention in D₂O at 600 MHz showing the negative (CH₂) signals

### EXAMPLES

### Example 1. Source and Extraction of Compound of the Present Invention

### 1.1. Fermentation of Streptromyces sp.

Streptomyces species were ordered from culture collections disclosed in Table 3. Streptomyces sp. Saigon413 was isolated in Vietnam before 1961. Streptomyces sp. Saigon413 was deposited at the Westerdijk institute under accession number CBS149411. The deposit was made by Syngenta Ltd., Jealott's Hill Research International Centre, Bracknell, Berkshire, RG42 6EY, UK under the terms of the Budapest Treaty on the International Recognition of the Deposit of Microorganisms for the Purposes of Patent Procedure.

The Streptomyces species were cultivated in Erlenmeyer flasks with a liquid medium consisting of (g/I) casein hydrolysate 10, glucose 40, K2HPO4 1.25, soytone 2, tryptone, 8 and incubated at 28 °C in an incubator shaking at 150 rpm with 25 mm throw for 4 days.

1.2. Isolation of 16S rDNA, and Species Identification

Genomic DNA was isolated from Streptomyces sp. Saigon413. using the method described in Kutchma et al. (1998) Biotechniques 24:452-457. The 16S rRNA gene was amplified using universal 16S primers and sequenced using Sanger sequencing. The 16S rRNA is shown in SEQ ID NO: 1. The species of strain Streptomyces sp. Saigon413 was identified by comparing the 16 S rRNA sequence according to SEQ ID NO:1 with publicly available 16S rRNA sequences that were extracted using whole genome sequence assembly of genomes from Streptomyces species (based on The Genome Taxonomy Database GTDB (Parks, D. H., et al. (2021). GTDB: Nucleic Acids Research, 50: D785-D794) using barrnap v0.9. Based on this comparative analysis Streptomyces sp. Saigon413 was identified as a Streptomyces chrestomyceticus species. The sequence identity between the 16S rRNA sequence of Streptomyces sp. Saigon413 and the publicly available S. chrestomyceticus NRRL-3672 was 99.87%, which was determined using Muscle v3.8.31 and R package Seqinr v4.2-16.

Whole genome sequencing, using the genomic DNA from Streptomyces sp. Saigon413, was completed using both Pacific Biosciences and Illumina sequencing technologies. The genome was assembled using HFAP4 and polished with Pilon using the Illumina reads. Genomic DNA was also extracted from Streptomyces rimosus CBS 492.64, Streptomyces rimosus CBS 570.66, Streptomyces rimosus CBS 569.66, Streptomyces chrestomyceticus DSM 41224, Streptomyces rimosus subsp. rimosus DSM 40673, and Streptomyces rimosus subsp. rimosus DSM 41057 using a method described in Kieser et. al., (2000) Practical Streptomyces Genetics. Whole genome sequencing for these strains was completed using Nanopore Sequencing technology and the genomes were assembled with Flye (Kolmogorov, M., et. al., (2019), Nature Biotechnology, 37, 540).

Following assembly of the genome from Streptomyces sp. Saigon 413 and publicly available genomes, the average nucleotide identity (ANI) was calculated between Streptomyces sp. Saigon413 and closely related Streptomyces strains using fastANI (Jain, C., et. al. (2018), Nature Communications, 9, 5114) (Table 3). The highest percentage identity (ANI) of the genome of of Streptomyces sp. Saigon413 was 96.9% with the genome of the publicly available S. chrestomyceticus NRRL B-3672.

Using the 16SRNA sequence identity and ANI score (%), it was also found that the strains CBS 596.66, CBS570.66 and DSM 41429 were Streptomyces chrestomyceticus strains and not a Streptomyces rimosis or Streptomyces paromomycinus strain as indicated by the depository institute.

### 1.3. Purification of the Compound of the Invention

Whole broth was centrifuged to produce an aqueous extract and a pellet. The aqueous extract was freeze dried. The material was resuspended in a minimal volume of water and partitioned with ethyl acetate to remove lipophilic components. The aqueous suspension was retained and freeze dried and resuspended in a minimal volume of water before being applied to an activated charcoal column. The column was washed with water and eluted with water:acetone (50:50).

The compound of the present invention was further purified by Hydrophilic Interaction Liquid Chromatography (HILIC) using Mass guided fractionation and ELSD detector. Using for example Waters XBridge Amide, 5 micron, 30×100 mm using a gradient of acetonitrile and 10 mM Ammonium Acetate.

### Example 2. Characterisation of Compound of the Present Invention

The compound of the present invention was determined in the purified fermentation broth according to the methods disclosed below.

The results in Table 3 show that several Streptomyces sp. produce the compound according to the present invention.

### 2.1. Molecular Composition and Total Molecular Mass

The molecular composition and total molecular mass was C₅₃H₉₀N₂O₄₄, and 1458.487 g, respectively which were determined using MS-MS and NMR spectroscopy as disclosed in paragraph 2.3 and 2.4.

### 2.2. Solubility

The solubility of the compound of the present invention was determined.

The solubility of the compound of the invention in water, pH 7.01, was >10′000 ppm, and in DMSO it was >9772 ppm.

### 2.3. MS-MS Spectrometry and Liquid Chromatography

Spectra were recorded on an Orbitrap ID-X Tribrid Mass Spectrometer from Thermo Scientific equipped with an OptaMax NG Heated Electrospray Source (Spray Voltage: Static, Polarity Ion (V): 3400 (Positive ion mode) & 2400 (Negative ion mode), Sheath Gas (Arb): 40, Aux Gas (Arb): 5, Sweep Gas (Arb): 1, lon Transfer Tube Temperature: 350 °C, Vaporizer Temperature: 350 °C). The Scan Parameters were as follows;
Experiment 1: MS OT (Orbitrap Resolution: 60,000, Scan Range (m/z): 200 to 2000, RF Lens (%): 60, AGC Target: Standard, Maximum Injection Time Mode: Auto, Microscans: 1, Data Type: Profile, Polarity: Both),
Experiment 2: tMS2 OT CID (MSn Level (n): 2, Isolation Window (m/z): 1.6, Activation Type: CID, CID Collision Energy (%): 30, Detector Type: Orbitrap, Orbitrap Resolution: 30,000, RF Lens (%): 60, Polarity: Negative). The mass spectrometer was connected to a Vanquish Flex UHPLC from Thermo Scientific using a Vanquish Split Sampler FT, Vanquish Binary Pump F, Vanquish Column Compartment H, Vanquish Diode Array Detector FG and Vanquish Charged Aerosol Detector. Liquid Chromatography Conditions included: Thermo Scientific Hypercarb™ Porous Graphitic Carbon column 5 μm 4.6×50 mm, P.N. 35005-054630. Temp: 40 °C, DAD wavelength range: 250 to 260 nm, Solvent gradient: Solvent A: H₂O with 0.1% formic acid, Solvent B: CH₃CN with 0.1% formic acid, gradient: 0 min 1% B, 99% A; 4.00 min 50% B, 50% A; 4.25 min 100% B; 4.50 min 100% B; 4.95 min 1% B, 99% A; 6.00 min 1% B, 99% A, Flow rate: 1.0 ml/min, Injection volume: 2 μL, Total run time: 6.0 min.

### 2.4. NMR Spectroscopy

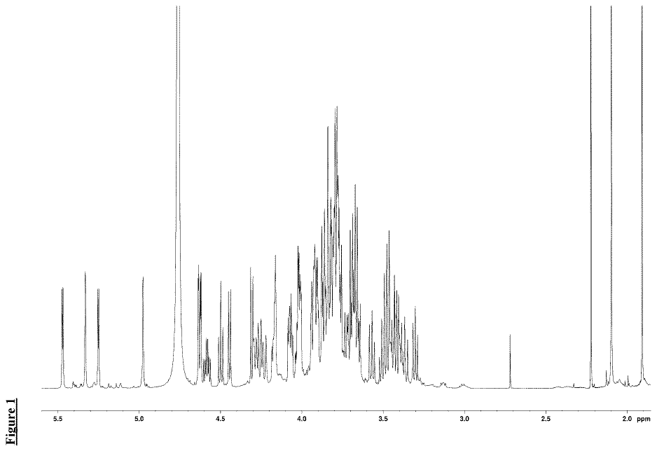

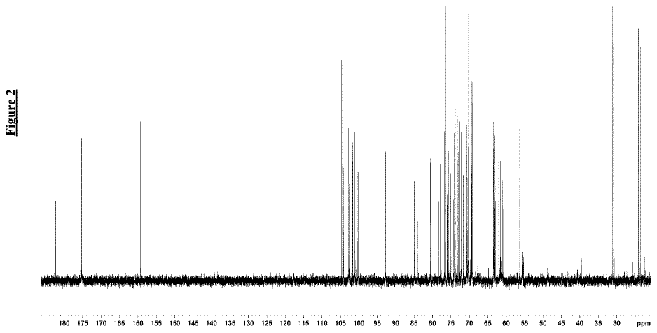

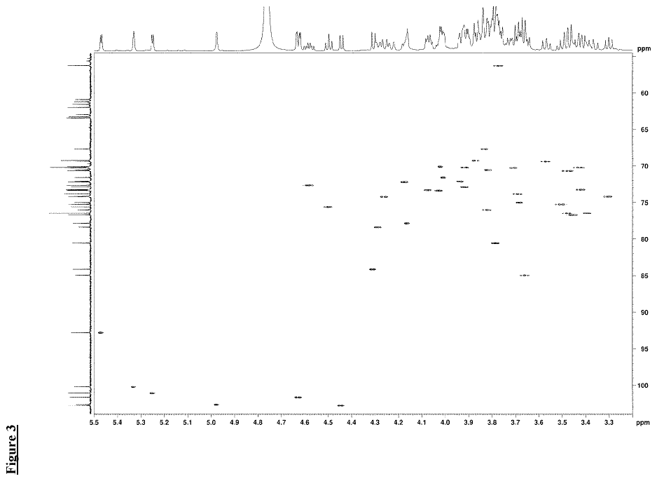

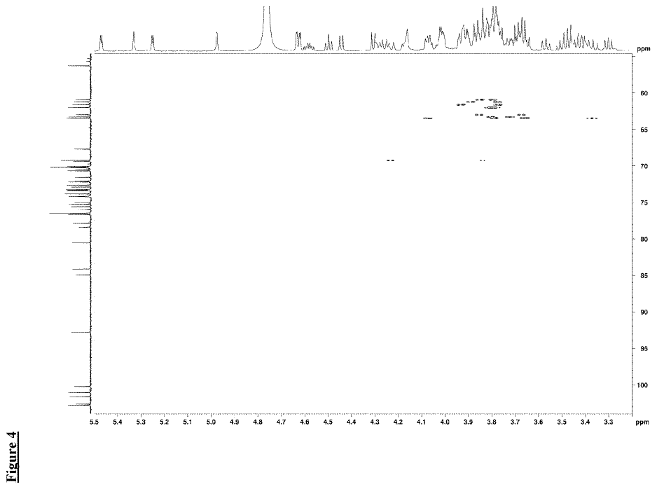

NMR spectra were recorded on a Bruker AVIII 600 NMR spectrometer, equipped with a 5 mm Bruker (¹H/¹⁹F)/¹³C/¹⁵N TCI cryoprobe fitted with Z gradients, using standard Bruker pulse sequences. Samples were dissolved in D₂O, and the spectra were recorded at 300° K and referenced to acetone at 2.225 ppm for ¹H and 31.07 ppm for ¹³C. FIGS. 1 to 4 show NMR spectra of the compound of the present invention.

The one bond ¹H—¹³C correlation spectrum contains peaks corresponding to 1 methyl (CH₃) and 40 methine (CH) groups (listed in Table 1) and 9 methylene (CH₂) groups (listed in Table 2).

In addition, the 1D ¹³C spectrum contains signals from 3 quaternary carbons at 104.7, 159.3 and 175.2 ppm (+0.1).

TABLE 1
Methyl and methine signals in the one bond ¹H—¹³C
correlation spectrum of a compound according to
the present invention together with multiplicity information
for protons resolved in the 1D ¹H spectrum.

| d ¹³C ppm (±0.1 ) | d ¹H ppm (±0.05) | ¹H Multiplicity |
|---|---|---|
| 104.2 | 4.632 | d (8.0, 1H) |
| 102.8 | 4.45 | d (7.5, 1H) |
| 102.6 | 4.98 | d (2.0, 1H) |
| 101.6 | 4.628 | d (8.5, 1H) |
| 101.1 | 5.25 | d (4.0, 1H) |
| 100.2 | 5.33 | d (2.0, 1H) |
| 92.8 | 5.47 | d (4.0, 1H) |
| 84.9 | 3.66 | |
| 84.1 | 4.31 | d (8.0, 1H) |
| 80.5 | 3.79 | |
| 78.4 | 4.29 | |
| 77.8 | 4.16 | |
| 76.7 | 3.46 | |
| 76.5 | 3.48 | |
| 76.5 | 3.39 | |
| 76.0 | 3.82 | |
| 75.6 | 4.50 | t (8.0, 1H) |
| 75.2 | 3.51 | |
| 75.0 | 3.68 | |
| 74.21 | 4.26 | |
| 74.18 | 3.30 | dd (8.0, 9.0, 1H) |
| 73.8 | 3.69 | |
| 73.4 | 4.03 | |
| 73.3 | 4.07 | |
| 73.2 | 3.42 | |
| 72.9 | 3.92 | |
| 72.6 | 4.58 | ddd (5.5, 9.5, 10.0, 1H) |
| 72.2 | 4.17 | |
| 72.1 | 3.94 | |
| 71.6 | 4.01 | |
| 70.7 | 3.48 | |
| 70.5 | 3.81 | |
| 70.3 | 3.71 | |
| 70.2 | 3.92 | |
| 70.2 | 3.43 | |
| 70.1 | 4.02 | |
| 69.4 | 3.57 | dd (9.0, 10.0, 1H) |
| 69.2 | 3.87 | |
| 67.7 | 3.84 | |
| 56.3 | 3.77 | |
| 23.6 | 2.10 | S (3H) |

TABLE 2
Methylene signals in the one bond ¹H—¹³C
correlation spectrum of a compound according to
the present invention together with multiplicity information
for protons resolved in the 1D ¹H spectrum.

| d ¹³C ppm (±0.1 ) | d ¹H ppm (±0.05) | ¹H Multiplicity |
|---|---|---|
| 69.2 | 4.23 | |
| 69.2 | 3.85 | |
| 63.5 | 4.07 | |
| 63.5 | 3.37 | dd (10.5, 11.5, 1H) |
| 63.4 | 3.79 | |
| 63.4 | 3.66 | |
| 63.3 | 3.81 | |
| 63.3 | 3.72 | |
| 63.0 | 3.85 | |
| 63.0 | 3.67 | |
| 62.0 | 3.82 | |
| 62.0 | 3.78 | |
| 61.6 | 3.93 | |
| 61.6 | 3.77 | |
| 61.2 | 3.89 | |
| 61.2 | 3.77 | |
| 60.9 | 3.85 | |
| 60.9 | 3.79 | |

TABLE 3
Identification of the compound of the invention in different Streptomyces
species and ANI (%) and 16SRNA % identities relative to S. sp. Saigon 413

| | | | ANI | | |
|---|---|---|---|---|---|
| | Species as | | (%) | 16SRNA | Compound of |
| strain ID | indicated by the | Species indicated by | Vs | sequence | the invention |
| (Collection) | source | ANI | CBS149411 | identity (%) | detected |
| CBS 149411 | Streptomyces sp. | Streptomyces | | | yes |
| (CBS) | Saigon413 | chrestomyceticus | | | |
| CBS 492.64 | S. rimosus | Streptomyces sp. | 90.04 | 99.48 | no |
| (CBS) | | | | | |
| CBS 569.66 | S. rimosus | Streptomyces | 99.74 | 99.94 | yes |
| (CBS) | | chrestomyceticus | | | |
| CBS 570.66 | S. rimosus | Streptomyces | 99.74 | 99.94 | yes |
| (CBS) | | chrestomyceticus | | | |
| CBS 612.68 | S. albofaciens | Streptomyces | 89.92 | 98.82 | yes |
| (CBS) | | albofaciens | | | |
| CBS 938.68 | S. rimosus subsp. | Streptomyces | 89.81 | 98.89 | yes |
| (CBS) | rimosus | rimosus | | | |
| DSM 40673 | S. rimosus | Streptomyces | 89.78 | 99.02 | yes |
| (DSMZ) | | rimosus | | | |
| DSM 41057 | S. rimosus | Streptomyces | 89.96 | 98.89 | No |
| (DSMZ) | | rimosus | | | |
| DSM 41224 | S. chrestomyceticus | Streptomyces | 96.96 | 99.87 | No |
| (DSMZ) | | chrestomyceticus | | | |
| DSM 41429 | S. paromomycinus | Streptomyces | 95.95 | 99.87 | yes |
| (DSMZ) | | chrestomyceticus | | | |
| DSM-41561 | S. rimosus subsp. | Streptomyces | 89.84 | 99.017 | No |
| (DSMZ) | rimosus | rimosus | | | |
| NRRL B-2626 | S. rimosus | Streptomyces | 89.75 | 98.89 | No |
| (ARS) | | rimosus | | | |
| NRRL B-3672 | S. chrestomyceticus | Streptomyces | 96.9 | 99.87 | yes |
| (ARS) | | chrestomyceticus | | | |
| NRRL B-3698 | S. rimosus | n.d. | n.d | n.d. | No |
| (ARS) | | | | | |
| NRRL WC-3869 | S. rimosus | Streptomyces | 89.87 | 98.95 | No |
| (ARS) | | rimosus | | | |

CBS Westerdijk fungal diversity institute: Uppsalalaan 8, 3584 CT, Utrecht, The Netherlands
DSMZ German Collection of Microorganisms and Cell Cultures: Inhoffenstraße 7B, 38124 Braunschweig, Germany.
ARS ARS Culture Collection (NRRL), 1815 N. University Street, Peoria, IL 61604, USA

### Example 3. Activity of the Compound According to the Present Invention on Plants

### 3.1. Leaf Disc or Leaf Segment Tests in Well Plates

Leaf disks or leaf segments of various plant species were cut from plants grown in the greenhouse. The cut leaf discs or segments were placed in multiwell plates (24-well format) onto water agar. The leaf disks were sprayed with a test solution before (preventative) or after (curative) inoculation. Compounds to be tested were prepared as water solutions (max. 10 mg/ml) which were diluted to the appropriate concentration with 0.025% Tween20 just before spraying. The inoculated leaf discs or segments were incubated under defined conditions (temperature, relative humidity, light, etc.) according to the respective test system. A single evaluation of disease level was carried out 3-9 days days after inoculation, depending on the pathosystem. Percent disease control relative to the untreated check leaf discs or segments was then calculated.

Puccinia recondita f. sp. tritici/Wheat/Leaf Disc Preventative (Brown Rust)

Wheat leaf segments cv. Kanzler were placed on agar in multiwell plates (24-well format) and sprayed with the formulated test compound diluted in water. The leaf disks were inoculated with a spore suspension of the fungus 1 day after application. The inoculated leaf segments were incubated at 19 °C and 75% rh under a light regime of 12 h light/12 h darkness in a climate cabinet and the activity of a compound was assessed as percent disease control compared to untreated leaf when an appropriate level of disease damage appeared in untreated check leaf segments (7-9 days after application).

Puccinia recondita f. sp. tritici/Wheat/Leaf Disc Curative (Brown Rust)

Wheat leaf segments cv. Kanzler were placed on agar in multiwell plates (24-well format). The leaf segments were inoculated with a spore suspension of the fungus. Plates were stored in darkness at 19 °C and 75% rh. The formulated test compound diluted in water was applied 1 day after inoculation. The leaf segments were incubated at 19 °C and 75% rh under a light regime of 12 h light/12 h darkness in a climate cabinet and the activity of a compound was assessed as percent disease control compared to untreated when an appropriate level of disease damage appeared in untreated check leaf segments (6-8 days after application).

Magnaporthe grisea (Pyricularia oryzae)/Rice/Leaf Disc Preventative (Rice Blast)

Rice leaf segments cv. Ballila were placed on agar in a multiwell plate (24-well format) and sprayed with the formulated test compound diluted in water. The leaf segments were inoculated with a spore suspension of the fungus 2 days after application. The inoculated leaf segments were incubated at 22 °C and 80% rh under a light regime of 24 h darkness followed by 12 h light/12 h darkness in a climate cabinet and the activity of a compound was assessed as percent disease control compared to untreated when an appropriate level of disease damage appeared in untreated check leaf segments (5-7 days after application).

TABLE 4
Reduction (%) of fungal development Puccinia recondita f. sp. tritici
preventive, Puccinia recondita f. sp. tritici curative and Magnaporthe grisea
preventive in the presence of several concentrations of the comound
of the invention on a leaf disc or leaf segment.

| | % reduction fungal growth | | |
|---|---|---|---|
| Plant leaf | wheat | wheat | rice |
| Compound of | Puccinia recondita f. | Puccinia recondita f. | Magnaporthe |
| the invention | sp. tritici | sp. tritici | grisea |
| (ppm) | preventive | curative | preventive |
| 200.00 | 95 | 100 | 100 |
| 67.00 | 45 | 100 | 70 |
| 22.00 | 0 | 90 | 60 |
| 7.40 | 0 | 0 | 0 |
| 2.50 | 0 | 0 | 0 |
| 0.80 | 0 | 0 | 0 |

The results in Table 4 show that the compound of the invention reduces fungal growth of several fungal species starting at a concentration of at 22 ppm in the leaf disc and leaf segment tests. The compound of the invention provides both preventive (application of compound followed by infection with pathogen) and curative (infection of pathogen followed by application of compound) activity to control the rust pathogen Puccinia recondita.

### 3.2. Leaf Painting Assay in Wheat Seedling

Wheat seedlings of the variety Riband (for Zymoseptoria tritici trials) or variety Arina (for Puccinia recondite trials) were grown in the glasshouse until 14d after seeding. At this time, such seedlings usually have a first leaf fully emerged (designated L1), a second leaf fully emerged (L2) and a third leaf (L3) that is partially emerged and in the process of development. Using a permanent pen, two dots were applied on the second leaf such as to generate three segments of roughly equal size: Segment A (at the base), segment B (middle part) and segment C (tip of the leaf). A stock solution of the compound of Formula (I) was produced in water and of benzovindiflupyr in DMSO at a concentration of 10,000 ppm. The stock solution was then further diluted in water supplemented with Tween20 to a final concentration of 200 ppm or higher of test compound, 0.05% Tween 20 and 2% DMSO (only for compounds with DMSO stock solution). The diluted compound was applied to the middle segment of L2 using a conventional cotton stick; the cotton stick was soaked in the diluted compound and rubbed several times on the adaxial leaf surface between the two marks. One day later, the complete plant was inoculated with a fungal spore suspension using a paint brush. The spore suspension was applied until before run-off.

For infection with Zymoseptoria tritici (preventive), the test plants were inoculated by spraying a spore suspension on them one day after application (1.5 Mio spores per ml in water supplemented with 0.01% Tween20). After an incubation period of 4 days at 22 °C/21 °C (day/night) and 95% rh, the inoculated test plants were kept at 22 °C/21 °C (day/night) and 70% rh in a greenhouse. Efficacy was assessed directly when an appropriate level of disease appeared on untreated check plants (16-19 days after application).

For infection with Puccinia recondita (preventive): The test plants were inoculated by spraying them with a spore suspension one day after application (spore suspension at 80,000 spores per ml in water supplemented with Tween20 at 0.1%). After an incubation period of 1 day at 20 °C and 95% rh, the inoculated test plants were kept at 20 °C and 60% rh in a greenhouse. The percentage leaf area covered by disease was assessed visually when an appropriate level of disease appeared on untreated check plants (9-12 days after infection).

The three segments of the leaf were evaluated individually.

TABLE 5
Reduction (%) of fungal growth of Puccinia recondita f. sp. tritici preventive
and Zymoseptoria tritici preventive on wheat leaves in the presence of the compound
of the present invention and benzovindiflupyr in a leaf painting assay

| | | | | % reduction fungal growth | |
|---|---|---|---|---|---|
| | | | | Puccinia | |
| | | | | recondita f. sp. | |
| | | | | tritici | Zymoseptoria tritici |
| | compound | ppm | segment | preventive | preventive |
| Experiment 1 | Compound of the | 200.00 | base | 0 | 24 |
| | invention | | | | |
| | | | middle | 48 | 93 |
| | | | tip | 31 | 99 |
| | benzovindiflupyr | 188.00 | base | 0 | 0 |
| | | | middle | 100 | 100 |
| | | | tip | 98 | 99 |
| | Tween20 0.05% | — | base | nt | 0 |
| | | | middle | nt | 0 |
| | | | tip | nt | 0 |
| | Tween20 0.05% and | — | base | nt | 0 |
| | 2% DMSO | | | | |
| | | | middle | nt | 0 |
| | | | tip | nt | 0 |
| Experiment 2 | Compound of the | 200.00 | base | 10 | nt |
| | invention | | | | |
| | | | middle | 79 | nt |
| | | | tip | 38 | nt |
| | benzovindiflupyr | 188.00 | base | 7 | nt |
| | | | middle | 100 | nt |
| | | | tip | 100 | nt |
| Experiment 3 | Compound of the | 400.00 | base | 16 | 1 |
| | invention | | | | |
| | | | middle | 98 | 96 |
| | | | tip | 99 | 100 |
| | benzovindiflupyr | 188.00 | base | 21 | 0 |
| | | | middle | 100 | 100 |
| | | | tip | 100 | 100 |
| Experiment 4 | Compound of the | 500.00 | base | 0 | nt |
| | invention | | | | |
| | | | middle | 69 | nt |
| | | | tip | 82 | nt |
| | benzovindiflupyr | 188.00 | base | 3 | nt |
| | | | middle | 100 | nt |
| | | | tip | 100 | nt |

nt.: not tested

The leaf painting tests show that the compound of the invention was active against growth of the fungi Puccinia recondite and Zymoseptoria tritici. The compound of the invention provided control on the treated area (middle segment) as well as the above treated area (tip). The activity identified in the plant was independent of the surfactant Tween 20 or the solvent DMSO that were also present at low concentration while testing a compound. The mobility of the efficacy towards an acropetal segment was similar to the SDHI fungicide benzovindiflupyr, which is well known to display such mobility.

### 3.3. Powdery Mildew Assay in Wheat

One-week-old wheat leaves were used for the experiment. 5 cm long sections from the leaf tip were cut under water and placed in cuvettes containing 100 μL of the test solution. After 1.5 h, leaves were removed and allowed to rest for 1 h on a paper towel. Then, leaves were placed on water agar plates (1%) and stored in a light cabinet (8 h of light at 18 °C). One day before inoculation of the harvested leaf sections, powdery mildew (Blumeria graminis f.sp. tritici) infected wheat plants were lightly shaken to remove older conidia to allow the formation of fresh conidia. For inoculation, the plates containing the leaf sections were laid out on the ground and subsequently an infection hood was placed over them. A powdery mildew infected wheat plant was held inside through an outlet in the hood and shaken gently to evenly distribute the spores. 48 h after inoculation, leaves were transferred to reaction tubes containing 10 mL of 80% ethanol. Ten days after decolorizing the leaf tissue, fungal spores were stained 10% (v/v) ink, 25% (v/v) acetic acid in ddH2O. At one hundred interaction sites of spores with plant cells, the ratio of successful penetrations versus papilla formation was assessed with a light microscope. The tests were repeated 4 times and the values indicated in Table 4 are the average of four tests. The compound INA (2.6-dichloro-isonicotinic acid, CAS: 5398-44-7) is a synthetic salicylic acid analog and was included as a reference for priming activity (Krauss et al., 1992, Plant Journal 2, 655-60).

The testsample AEF1 is a sample enriched for compound of Formula I. It corresponds to the fraction eluted from the activated charcoal column with the highest enrichment for compound of Formula I.

TABLE 6
Haustoria formation by Blumeria graminis f.sp. tritici
48 h after infecting wheat leaves treated with different
compounds. Values reported are haustorium frequency relative
to the mean control value. Different letters indicate significant
difference of the results (P-value < 0.05)

| | | haustoria | |
|---|---|---|---|
| | | formation (% | |
| | Concentration | relative to | |
| sample | (μM or ppm) | control) | Letter |
| Water (control) | — | 100.0 | a |
| INA (μM) | 100 | 27.6 | c |
| compound of formula I (μM) | 100 | 66.4 | b |
| | 25 | 59.1 | b |
| | 1 | 105.5 | a |
| AEF1 (ppm) | 100 | 72.7 | b |
| (Aqueous extract fraction 1) | 25 | 107.3 | a |
| | 1 | 106.4 | a |

The results in Table 6 demonstrate that wheat pre-treated with a compound of the present invention or a fraction enriched by the compound of the present invention resulted in reduction of haustoria formation from the powder mildew fungus Blumeria graminis f.sp. tritici. The effect is observed at a concentration of 25 μM or higher. The enriched fraction and the purified compound have a similar effect at the highest tested rates.

3.4. Reactive Oxygen Species (ROS) Burst Assay with Wheat Leaf Discs

200 μL of the test solution (analyte) or the respective control (water) in the appropriate concentration was pipetted into a white 96-well plate (Nunc, Langenselbold, Germany). 5 mm leaf discs from 2-week-old wheat plants were obtained using a tissue punch and subsequently floated on the test solution. The plates were stored for 24 h at RT. The next day, the solution was replaced with 50 μL ddH2O and leaf discs were left for regeneration for at least one hour at RT in the dark. Meanwhile appropriate master mixes, either with or without the elicitor flg22 (see below), were freshly prepared in black 5 mL reaction tubes. After regeneration, 50 μL of the corresponding master mix was added to the leaf disc containing wells. Subsequently, luminescence was recorded for 40 minutes with a platereader (BMG Labtech; Ortenberg, Germany). The compound INA (2.6-dichloro-isonicotinic acid, CAS: 5398-44-7) is a synthetic salicylic acid analog and was included as a reference for priming activity (Kauss et al., 1992).
Mastermix −flg22: 4.98 mL ddH2O, 10 μL HRP (10 mg/mL), 10 μL L-012 (20 mM)
Mastermix +flg22: 4.979 mL ddH2O, 10 μL HRP (10 mg/mL), 10 μL L-012 (20 mM), 1 μL flg22 (10 μM) Abbreviations: flg22 (22 amino acids flagellin peptide. Eurogentec Cat. Number AS-62633); HRP (Horseradish Peroxidase), used L-012 sodium salt (CAS #: 143556-24-5).

The testsample AEF1 is a sample enriched for compound of Formula (I). It corresponds to the fraction eluted from the activated charcoal column with the highest enrichment for compound of Formula I.

TABLE 7
Fold change of peak ROS production. Numbers report the ratio
of peak values observed for treated vs. control, using the
maximum value each condition measured over a time-course of
40 minutes. Values indicated are the mean of two repetitions.

| | Concentration | Fold change |
|---|---|---|
| Sample | (ppm) | (peak ROS) |
| Water (control) | — | 1.0 |
| INA (μM) | 100 | 1.8 |
| AEF1 (ppm) | 100 | 3.0 |
| (Aqueous extract fraction 1) | 25 | 2.1 |
| | 1 | 2.0 |

The results in Table 7 demonstrate that wheat pretreated with a fraction enriched with a compound of the preseng invention in a concentration range from 1 ppm to 100 ppm increased ROS production induced by the peptide flg22 by a factor of 2 or higher. This is a response that is similar to, or stronger than the response observed from a treatment with INA (2.6-dichloro-isonicotinic acid), a well-known priming agent (Krauss et al., 1992, Plant Journal 2:655-60).

### Example 4. Fungicidal Activity of a Mixture of the Oligosaccharide Compound According to the Present Invention and Malonomicin in Leaf Disc Assays

A stock solution for oligosaccharide compound according to the present invention was produced in water plus 0.025% Tween®20. Malonomicin was produced according to Law et al, 2018 (Nature Catalys is | VOL 1 | DECEMBER 2018 | 977-984). A stock solution of malonomicin was produced in water plus 0.025% Tween®20.

Assays to test efficacy of mixtures of the compound of Formula (I) in combination with malonomicin on the control of fungal pathogens in a leaf disc assay were designed for two 24 well plates.

Table 8 and 9: Outline of 24 well testplate (1) and testplate (2) including concentration of compounds sprayed per well. The upper number in the cell indicates the oligosaccharide compound of the present invention concentration (ppm), the lower number malonomicin concentration (ppm). Column (1) holds a dilution series of compound of Formula (I), row (2-D) hold a dilution series of malonomicin. Well 2-D-(1) represents an untreated check. This design was applied for tests including Puccinia recondita (EPPO code: PUCCRE) with preventive and curative spray timing.

TABLE 8
24 well plate (1)

| ppm | (1) | (2) | (3) | (4) | (5) | (6) |
|---|---|---|---|---|---|---|
| 1-A | 200 | 200 | 200 | 200 | 200 | 200 |
| | 0 | 200 | 20 | 6.67 | 2.22 | 0.74 |
| 1-B | 100 | 100 | 100 | 100 | 100 | 100 |
| | 0 | 200 | 20 | 6.67 | 2.22 | 0.74 |
| 1-C | 50 | 50 | 50 | 50 | 50 | 50 |
| | 0 | 200 | 20 | 6.67 | 2.22 | 0.74 |
| 1-D | 25 | 25 | 25 | 25 | 25 | 25 |
| | 0 | 200 | 20 | 6.67 | 2.22 | 0.74 |

TABLE 9
24-well plate (2)

| ppm | (1) | (2) | (3) | (4) | (5) | (6) |
|---|---|---|---|---|---|---|
| 2-A | 12.5 | 12.5 | 12.5 | 12.5 | 12.5 | 12.5 |
| | 0 | 200 | 20 | 6.67 | 2.22 | 0.74 |
| 2-B | 6.25 | 6.25 | 6.25 | 6.25 | 6.25 | 6.25 |
| | 0 | 200 | 20 | 6.67 | 2.22 | 0.74 |
| 2-C | 3.13 | 3.13 | 3.13 | 3.13 | 3.13 | 3.13 |
| | 0 | 200 | 20 | 6.67 | 2.22 | 0.74 |
| 2-D | 0 | 0 | 0 | 0 | 0 | 0 |
| | 0 | 200 | 20 | 6.67 | 2.22 | 0.74 |

TABLE 10
Mixture ratio of compounds sprayed in the 24well plate assay
as outlined in Table 8 and 9. The number represents the
ratio of compound 1:compound 2. Compound 1 is malonomicin,
and compound 2 is the oligosaccharide compound of the present
invention. The plate design across the two 24well plates
spans a wide range from 64:1 up to 1:270.

| | | | | | |
|---|---|---|---|---|---|
| (1) | (2) | (3) | (4) | (5) | (6) |

| | | | | | |
|---|---|---|---|---|---|
| 1-A | 1:1 | 1:10 | 1:30 | 1:90 | 1:270 |
| 1-B | 2:1 | 1:5 | 1:15 | 1:45 | 1:135 |
| 1-C | 4:1 | 1:2.5 | 1:7.5 | 1:23 | 1:68 |
| 1-D | 8:1 | 1:1.3 | 1:3.7 | 1:11 | 1:34 |
| 2-A | 16:1 | 1.6:1 | 1:1.9 | 1:5.6 | 1:17 |
| 2-B | 32:1 | 3.2:1 | 1.1:1 | 1:2.8 | 1:8.4 |
| 2-C | 64:1 | 6.4:1 | 2.1:1 | 1:1.4 | 1:4.2 |
| 2-D | | | | | |

Table 11 and Table 12: Outline of 24 well testplate (3) and testplate (4) including concentration of compounds per well. The upper number in the cell indicates the oligosaccharide compound of the present invention concentration (ppm), the lower number malonomicin concentration (ppm). Column (1) holds a dilution series of compound of Formula (I), row (4-D) hold a dilution series of malonomicin. Well 4-D-(1) represents an untreated check. This design was applied for tests including Blumeria graminis f.sp. tritici (EPPO code: ERYSGT) with preventive spray timing and Parastagonospora nodorum (EPPO code: LEPTNO) with preventive spray timing.

TABLE 11
24-well plate (3)

| ppm | (1) | (2) | (3) | (4) | (5) | (6) |
|---|---|---|---|---|---|---|
| 3-A | 600 | 600 | 600 | 600 | 600 | 600 |
| | 0 | 600 | 200 | 66.7 | 22.2 | 7.4 |
| 3-B | 300 | 300 | 300 | 300 | 300 | 300 |
| | 0 | 600 | 200 | 66.7 | 22.2 | 7.4 |
| 3-C | 150 | 150 | 150 | 150 | 150 | 150 |
| | 0 | 600 | 200 | 66.7 | 22.2 | 7.4 |
| 3-D | 75 | 75 | 75 | 75 | 75 | 75 |
| | 0 | 600 | 200 | 66.7 | 22.2 | 7.4 |

TABLE 12
24 well plate (4)

| ppm | (1) | (2) | (3) | (4) | (5) | (6) |
|---|---|---|---|---|---|---|
| 4-A | 37.5 | 37.5 | 37.5 | 37.5 | 37.5 | 37.5 |
| | 0 | 600 | 200 | 66.7 | 22.2 | 7.4 |
| 4-B | 18.75 | 18.75 | 18.75 | 18.75 | 18.75 | 18.75 |
| | 0 | 600 | 200 | 66.7 | 22.2 | 7.4 |
| 4-C | 9.38 | 9.38 | 9.38 | 9.38 | 9.38 | 9.38 |
| | 0 | 600 | 200 | 66.7 | 22.2 | 7.4 |
| 4-D | 0 | 0 | 0 | 0 | 0 | 0 |
| | 0 | 600 | 200 | 66.7 | 22.2 | 7.4 |

TABLE 13
Mixture ratio of compounds sprayed in the 24well plate assay
as outlined in Table 11 and Table 12. The number represents
the ratio of compound 1:compound 2. Compound 1 is malonomicin,
and compound 2 is the compound of Formula (I) according to
the present invention. The plate design across the two 24well
plates spans a wide range from 64:1 up to 1:81.

| | | | | | |
|---|---|---|---|---|---|
| (1) | (2) | (3) | (4) | (5) | (6) |

| | | | | | |
|---|---|---|---|---|---|
| 3-A | 1:1 | 1:3 | 1:9 | 1:27 | 1:81 |
| 3-B | 2:1 | 1:1.5 | 1:4.5 | 1:14 | 1:41 |
| 3-C | 4:1 | 1.3:1 | 1:2.2 | 1:6.8 | 1:20 |
| 3-D | 8:1 | 2.7:1 | 1:1.1 | 1:3.4 | 1:10 |
| 4-A | 16:1 | 5.3:1 | 1.8:1 | 1:1.7 | 1:5.1 |
| 4-B | 32:1 | 11:1 | 3.6:1 | 1.2:1 | 1:2.5 |
| 4-C | 64:1 | 21:1 | 7.1:1 | 2.4:1 | 1:1.3 |
| 4-D | | | | | |

A first set of master plates with 1× concentrated spray solution of the compound of Formula (I) stock diluted in water with concentrations according to Table 8, Table 9, Table 11 and Table 12, respectively, was prepared. Each well contained 0.025% Tween®20. Accordingly, a second set of master plates with 1× concentrated malonomicin stock diluted in water was prepared. Each well of the second set contained 0.025% Tween®20. Leaf segments placed on agar in 24 well plates are sprayed with 8 ul of solution from the master plate containing compound of Formula (I) and the leaf segments were let to dry, followed 2 hours later by 8 ul of solution from the master plate containing malonomicin. After second sprays have dried, leaf segments are infected with fungal spores to obtain a preventive application timing. Alternatively, leaf segments infected one day before spray of the compounds are used, resulting in a curative spray timing. In addition, several plates were produced where leaf segments were sprayed 2× in absence of test compound (with only Tween®20), representing the untreated check samples. For each leaf segment percent leaf coverage of disease symptoms was assessed. Percent leaf coverage reduction relative to the untreated check was calculated. Efficacy of the mixtures was tested in duplicate and on different fungal species. Reported efficacy values are the average of two replicate results.

Puccinia recondita (EPPO Code: PUCCRE) with Preventive Spray Timing.

Wheat (cultivar Kanzler) leaf segments are placed on agar in multiwell plates (24-well format) and sprayed with test solutions (8 ul per well). After drying, the leaf disks are inoculated with a spore suspension of the fungus. After appropriate incubation the activity of a compound is assessed 8 dpi (days after inoculation) as preventive fungicidal activity.

Puccinia recondita (EPPO Code: PUCCRE) with Curative Spray Timing.

Wheat (cultivar Kanzler) leaf segments are placed on agar in multiwell plates (24-well format). The leaf disks are then inoculated with a spore suspension of the fungus. One day after inoculation the test solution is sprayed (8 ul per well). After appropriate incubation the activity of a compound is assessed 8 dpi (days after inoculation) as curative fungicidal activity

Blumeria graminis f.sp. tritici (EPPO Code: ERYSGT) with Preventive Spray Timing

Wheat (cultivar Kanzler) leaf segments are placed on agar in multiwell plates (24-well format) and sprayed with test solutions (8 ul per well). After drying, the leaf disks are inoculated with spores of the fungus. After appropriate incubation the activity of a compound is assessed 7 dpi (days post inoculation) as preventive fungicidal activity.

Parastagonospora nodorum (EPPO Code: LEPTNO)

Wheat (cultivar Kanzler) leaf segments are placed on agar in multiwell plates (24-well format) and sprayed with test solutions (8 ul per well). After drying, the leaf disks are inoculated with a spore suspension of the fungus. After appropriate incubation the activity of a compound is assessed 4 dpi (days after inoculation) as preventive fungicidal activity.

### Results

The results of the fungal control experiments with a mixture of the compound according to the present invention and malonomicin described above are shown in Tables 14 to 19.

TABLE 14
Control of Puccinia recondita (preventive) for solo compounds
and mixtures. The plate design including the concentration of the
compound of Formula (I) and malonomicin are indicated in Table 8
and Table 9. Values indicate control of fungal growth (% reduction
of symtoms on the leaf segment as compared to untreated check).

| | (1) | (2) | (3) | (4) | (5) | (6) |
|---|---|---|---|---|---|---|
| 1-A | 60 | 100 | 100 | 90 | 60 | 25 |
| 1-B | 10 | 100 | 100 | 95 | 70 | 0 |
| 1-C | 25 | 100 | 100 | 95 | 25 | 10 |
| 1-D | 10 | 100 | 100 | 80 | 50 | 0 |
| 2-A | 10 | 100 | 100 | 80 | 35 | 10 |
| 2-B | 10 | 100 | 100 | 80 | 35 | 0 |
| 2-C | 0 | 100 | 100 | 90 | 35 | 10 |
| 2-D | 10 | 100 | 100 | 80 | 10 | 0 |

For each of the assay conditions in Table 14 with efficacy of 50% or more (effective mixture) the respective mixture ratio can be assigned from Table 10. Effective mixtures for the control of Puccinia recondita (preventive) were found from 64:1 to 1:90.

TABLE 15
Comparison of measured values for disease control of Puccinia recondita
(preventive) (as reported in Table 14) with calculated values using
the formula from Colby for the same mixture. Numbers reported in the
table represent the difference from measured efficacy (in %) minus
calculated efficacy (in %). Values around 0 (zero) indicate additive
activity, while positive values suggest synergistic activity.

| | | | | | |
|---|---|---|---|---|---|
| (1) | (2) | (3) | (4) | (5) | (6) |

| | | | | | |
|---|---|---|---|---|---|
| 1-A | 0 | 0 | −2 | −4 | −35 |
| 1-B | 0 | 0 | 13 | 51 | −10 |
| 1-C | 0 | 0 | 10 | −8 | −15 |
| 1-D | 0 | 0 | −2 | 31 | −10 |
| 2-A | 0 | 0 | −2 | 16 | 0 |
| 2-B | 0 | 0 | −2 | 16 | −10 |
| 2-C | 0 | 0 | 10 | 25 | 10 |
| 2-D | | | | | |

TABLE 16
Control of Puccinia recondita (curative) for solo compounds and
mixtures. The plate design including the concentration of compound
of Formula (I) and malonomicin are indicated in Table 8 an dTable
8. Values indicate control of fungal growth (% reduction of symtoms
on the leaf segment as compared to untreated check).

| | (1) | (2) | (3) | (4) | (5) | (6) |
|---|---|---|---|---|---|---|
| 1-A | 80 | 100 | 100 | 100 | 90 | 80 |
| 1-B | 70 | 100 | 100 | 100 | 95 | 70 |
| 1-C | 20 | 100 | 100 | 100 | 95 | 60 |
| 1-D | 10 | 100 | 100 | 100 | 90 | 60 |
| 2-A | 10 | 100 | 100 | 100 | 95 | 60 |
| 2-B | 0 | 100 | 100 | 100 | 85 | 10 |
| 2-C | 0 | 100 | 100 | 100 | 90 | 35 |
| 2-D | 10 | 100 | 100 | 100 | 70 | 35 |

For each of the assay conditions in Table 16 with efficacy of 50% or more (effective mixture) the respective mixture ratio can be assigned from Table 10. Effective mixtures for the control of Puccinia recondita (curative) were found from 64:1 to 1:270.

TABLE 17
Comparison of measured values for disease control of Puccinia recondita
(curative) (as reported in Table 16) with calculated values using
the formula from Colby for the same mixture. Numbers reported in the
table represent the difference from measured efficacy (in %) minus
calculated efficacy (in %). Values around 0 (zero) indicate additive
activity, while positive values suggest synergistic activity.

| | | | | | |
|---|---|---|---|---|---|
| (1) | (2) | (3) | (4) | (5) | (6) |

| | | | | | |
|---|---|---|---|---|---|
| 1-A | 0 | 0 | 0 | −4 | −7 |
| 1-B | 0 | 0 | 0 | 4 | −11 |
| 1-C | 0 | 0 | 0 | 19 | 12 |
| 1-D | 0 | 0 | 0 | 17 | 19 |
| 2-A | 0 | 0 | 0 | 22 | 19 |
| 2-B | 0 | 0 | 0 | 15 | −25 |
| 2-C | 0 | 0 | 0 | 20 | 0 |
| 2-D | | | | | |

TABLE 18
Control of Blumeria graminis f. sp. tritici (preventive)
for solo compounds and mixtures. The plate design including
the concentration of compound of Formula (I) and malonomicin
are indicated in Table 11 and Table 12. Values indicate
control of fungal growth (% reduction of symtoms on the
leaf segment as compared to untreated check).

| | (1) | (2) | (3) | (4) | (5) | (6) |
|---|---|---|---|---|---|---|
| 3-A | 10 | 95 | 70 | 35 | 25 | 10 |
| 3-B | 10 | 95 | 95 | 50 | 25 | 0 |
| 3-C | 0 | 95 | 90 | 10 | 10 | 0 |
| 3-D | 20 | 95 | 100 | 25 | 35 | 0 |
| 4-A | 0 | 100 | 100 | 70 | 35 | 10 |
| 4-B | 0 | 95 | 90 | 50 | 25 | 0 |
| 4-C | 10 | 85 | 95 | 35 | 10 | 0 |
| 4-D | 0 | 75 | 100 | 70 | 0 | 0 |

For each of the assay conditions in table 18 with efficacy of 50% or more (effective mixture) the respective mixture ratio can be assigned from Table 11. Effective mixtures for the control of Blumeria graminis f.sp. tritici (preventive) were found from 64:1 to 1:4.

TABLE 19
Control of Parastagonospora nodorum (preventive) for solo compounds
and mixtures. The plate design including the concentration of compound
of Formula (I) and malonomicin are indicated in Table 11 and 12.
Values indicate control of fungal growth (% reduction of symtoms
on the leaf segment as compared to untreated check).

| | (1) | (2) | (3) | (4) | (5) | (6) |
|---|---|---|---|---|---|---|
| 3-A | 0 | 95 | 95 | 70 | 20 | 0 |
| 3-B | 0 | 100 | 95 | 70 | 35 | 10 |
| 3-C | 0 | 85 | 95 | 45 | 0 | 0 |
| 3-D | 0 | 100 | 90 | 55 | 10 | 0 |
| 4-A | 0 | 100 | 90 | 90 | 45 | 10 |
| 4-B | 0 | 100 | 95 | 80 | 20 | 10 |
| 4-C | 0 | 90 | 90 | 60 | 20 | 10 |
| 4-D | 0 | 95 | 90 | 50 | 45 | 0 |

For each of the assay conditions in Table 19 with efficacy of 50% or more (effective mixture) the respective mixture ratio can be assigned from Table 11. Effective mixtures for the control Parastagonospora nodorum (preventive) were found from 64:1 to 1:9.

### CONCLUSION

Mixtures of the oligosaccharide compound according to the present invention and malonomicin control various fungal pathogens. A large variety of mixture ratio's of the two compounds in the mixtures still produce 50% or more control of the fungal growth when sprayed on a leaf. Examples are shown for ratio's that range from 64:1 to 1:270, for several fungal species, including examples with a ratio in between the mentioned ratios (ratio of compounds malonomicin:oligosaccharide compound of the present invention). Surprisingly, the efficacy of several mixtures is better than the efficacy predicted based on the calculation from Colby, indicating that the mixture of the oligosaccharide compound of the invention with malonomicin has a synergistic effect on the control of fungal pathogens when sprayed on a leaf. This surprising synergistic effect was observed in tests to control Puccinia recondita.

1. A compound comprising a molecular formula according to C₅₃H₉₀N₂O₄₄, further characterised by the NMR spectra listed in Table 1 and Table 2, or a salt thereof.

2. A compound, optionally according to claim 1, having the Structural Formula (I): 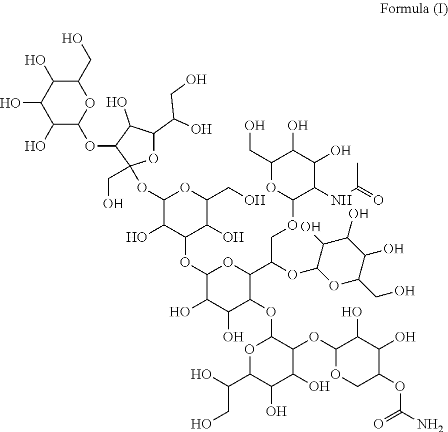 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]
or a salt thereof.

3. A composition comprising the compound according to claim 1 and a microorganism that is able to produce a compound thereof.

4. A composition comprising the compound according to claim 1 or the composition thereof, further comprising an auxiliary.

5. The compound according to claim 1 or the composition thereof, wherein the compound or the composition has fungicidal activity.

6. The composition according to claim 3, further comprising at least one additional ingredient having pesticidal activity and/or at least one plant growth regulator.

7. The composition according to claim 6, wherein the additional ingredient comprises cyclothiazomycin C, streptimidone and/or malonomicin, preferably malonomicin

8. A process for producing the compound according to claim 1, or the composition thereof comprising cultivating a microorganism in a suitable fermentation medium under conditions that allow production of the compound.

9. The composition according to claim 3, or the process thereof, wherein the microorganism is a Streptomyces sp., preferably a Streptomyces chrestomyceticus, S. rimosus, S. paromomycinus, or S. albofaciens, preferably a Streptomyces sp. which has a 16S RNA sequence which has at least 98% identity to SEQ ID NO: 1, preferably wherein the microorganism is Streptomyces sp. Saigon413 deposited with the Westerdijk Institute under accession number CBS149411.

10. A method for controlling or preventing infestation of a plant, plant propagation material, a locus and/or harvested food crops by a phytopathogenic microorganism, by treating the plant, plant propagation material, locus and/or harvested food crops, wherein an effective amount of the compound according to claim 1 or the composition thereof is applied to the plant, to a part thereof, the plant propagation material, the locus thereof and/or harvested food crops.

11. The method according to claim 10, wherein the effective amount comprises 5 g to 5 kg of the compound thereof per hectare.

12. The method according to claim 10, wherein the plant propagation material is seed and the effective amount comprises 0.001 to 50 g of the compound thereof per kg of seed.

13. The method according to claim 10, wherein the phytopathogenic microorganism is a fungus, preferably belonging to Zymoseptoria, Puccinia, Magnaporthe, Blumeria or Parastagonospora, preferably a fungus belonging to Zymoseptoria tritici, Puccinia recondite, Puccinia recondita f. sp. tritici, Magnaporthe grisea, Blumeria graminis f.sp. tritici, or Parastagonospora nodorum.

14. The method according to claim 10, wherein the plant is rice, wheat, corn, soya, or banana.

15. Use of a compound according to claim 1 or a composition thereof as a pesticide, preferably as a fungicide, and/or as a priming agent.
